(19) United States
(12) Patent Application Publication
(10) Pub. No.: US 2013/0267522 A1
(43) Pub. Date: Oct. 10, 2013

(54) METHODS FOR TREATING CANCER

(71) Applicants: Deborah Knapp, West Lafayette, IN (US); Deepika Dhawan, West Lafayette, IN (US); Philip S. Low, West Lafayette, IN (US); Paul Joseph Kleindl, Lebanon, IN (US); Iontcho R. Vlahov, West Lafayette, IN (US)

(72) Inventors: Deborah Knapp, West Lafayette, IN (US); Deepika Dhawan, West Lafayette, IN (US); Philip S. Low, West Lafayette, IN (US); Paul Joseph Kleindl, Lebanon, IN (US); Iontcho R. Vlahov, West Lafayette, IN (US)

(73) Assignees: Endocyte, Inc., West Lafayette, IN (US); Purdue Research Foundation, West Lafayette, IN (US)

(21) Appl. No.: 13/779,501

(22) Filed: Feb. 27, 2013

Related U.S. Application Data
(60) Provisional application No. 61/605,678, filed on Mar. 1, 2012.

(51) Int. Cl. A61K 31/519

(52) U.S. Cl. CPC A61K 31/519
USPC 514/249; 435/7.23

(57) ABSTRACT

The invention relates to methods and uses for the treatment of a cancer. In particular, the invention relates to the use of a folate-vinca conjugate to treat urinary bladder cancer (e.g., invasive transitional cell carcinoma (InvTCC)).

### RELATED APPLICATIONS

This application claims the benefit of U.S. Provisional Application No. 61/605,678, filed Mar. 1, 2012, the content of which is incorporated by reference.

### TECHNICAL FIELD

The invention relates to methods for the treatment of a urinary bladder cancer. In particular, the invention relates to methods for the treatment of a urinary bladder cancer with a particular folate-vinca conjugate.

### BACKGROUND

Invasive urinary bladder cancer kills more than 14,000 people each year in the United States. Most of those deaths are due to intermediate to high grade invasive transitional cell carcinoma (InvTCC) that has metastasized and is resistant to chemotherapy. Nonspecific cytotoxic drugs used to treat InvTCC have failed to eradicate the cancer or to provide long term control, and have caused substantial toxicity in many patients. The development of targeted therapy for InvTCC offers the opportunity to reduce treatment related toxicity and to increase treatment efficacy.

One solution to current chemotherapy limitations would be to deliver a biologically effective concentration of anti-cancer agents to the tumor tissues with very high specificity. To reach this goal, much effort has been undertaken to develop tumor-selective drugs by conjugating anti-cancer drugs to such ligands as hormones, antibodies, or vitamins. For example, the low molecular weight vitamin compound, folate, is useful as a tumor-targeting agent.

Folate is a member of the B family of vitamins and plays an essential role in cell survival by participating in the biosynthesis of nucleic acids and amino acids. This essential vitamin is also a high affinity ligand that enhances the specificity of conjugated anti-cancer drugs by targeting folate receptor (FR)-positive cancer cells. The FR, a tumor-associated glycosylphosphatidylinositol anchored protein, can actively internalize bound folates and folate conjugated compounds via receptor-mediated endocytosis. The FR is upregulated in more than 90% of non-mucinous ovarian carcinomas. The FR is also found at high to moderate levels in kidney, brain, lung, and breast carcinomas, while it occurs at low levels in most normal tissues. The FR density also appears to increase as the stage of the cancer becomes more advanced.

Although the overexpression of the FR has been documented in several forms of cancer in humans, its expression in InvTCC has not been previously reported. As described herein, FR expression has been shown in human InvTCC, as well as in a highly relevant animal model of InvTCC and in naturally-occurring InvTCC in dogs. InvTCC in dogs and humans is very similar in histopathology, molecular features, biological behavior including local invasion and distant metastasis, and response to chemotherapy. The naturally-occurring dog model of InvTCC is described in Knapp et al., “Animal Models: naturally occurring canine urinary bladder cancer,” In: Lerner et al., eds., Textbook of Bladder Cancer, Taylor Francis, Oxon, United Kingdom, 2006, pp. 171-175, and Knapp et al., “Naturally-occurring canine transitional cell carcinoma of the urinary bladder: A relevant model of human invasive bladder cancer,” Urol. Oncol., 47-59, 2000, both incorporated herein by reference.

### SUMMARY

Accordingly, the present invention relates to the development of folate-targeted therapy to treat cancer, including urinary bladder cancers (e,g, invasive transitional cell carcinoma (InvTCC; intermediate to high grade invasive) and low grade superficial urinary bladder cancer). In one embodiment, a method of treatment of a cancer is provided, comprising the step of administering EC0905, a folate-targeted conjugate, to a cancer patient in need thereof. In another embodiment, the cancer is urinary bladder cancer. In one example embodiment, the urinary bladder cancer treated is invasive transitional cell carcinoma (InvTCC). EC0905 is a compound of the formula:

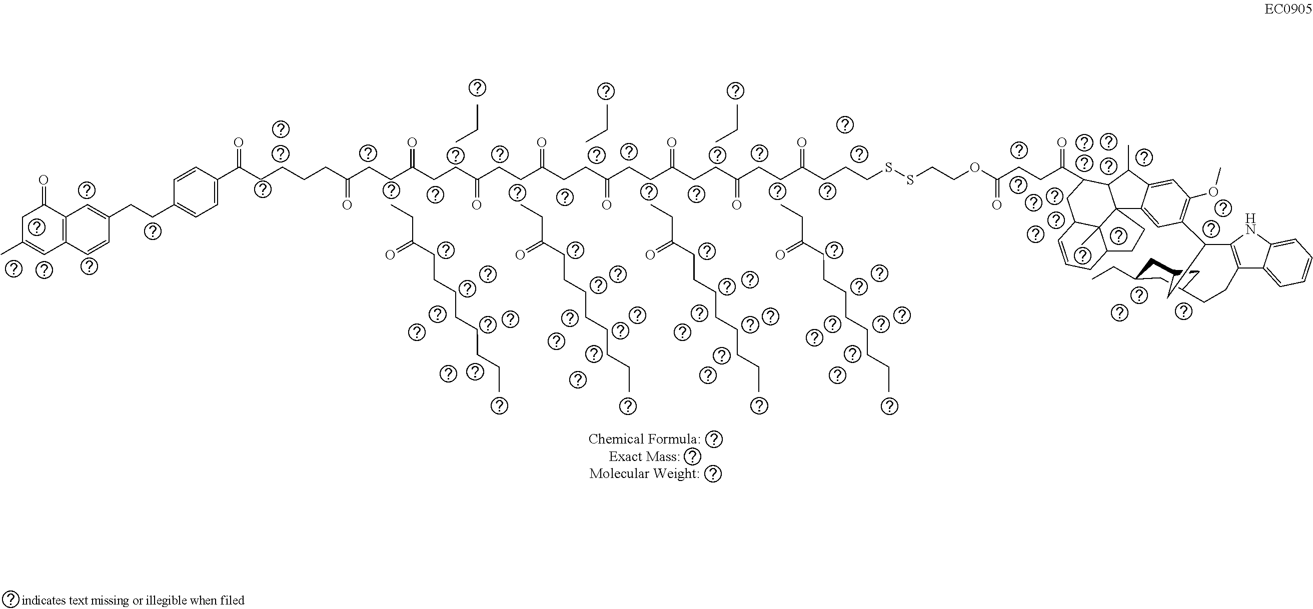

As used herein, the term “EC0905” means the chemotherapeutic agent, the structure of which is shown above, or a pharmaceutically acceptable salt thereof. The chemotherapeutic agent may be present in solution or suspension in an ionized form, including a protonated form. EC0905 can be synthesized, for example, by the method described in Examples 2-3, “EC0905” is used interchangeably with the term “conjugate” herein,

In one example embodiment, the EC0905 is in a composition and the composition further comprises a pharmaceutically acceptable carrier. In one example embodiment, the pharmaceutically acceptable carrier comprises a liquid carrier, In some embodiments, the liquid carrier is saline, glucose, an alcohol, a glycol, an ester, an amide, or a combination thereof.

In other embodiments, the compound or the composition is an inhalation dosage form, an oral dosage form, or a parenteral dosage form. In one example embodiment, the parenteral dosage form is an intradermal dosage form, a subcutaneous dosage form, an intramuscular dosage form, an intraperitoneal dosage form, an intravenous dosage form, or an intrathecal dosage form.

In another example embodiment, the compound or the composition is in the form of a solid. In some embodiments, the purity of the compound is at least 90%, 95%, 98%, or 99% based on weight percent.

In one example embodiment, use of a compound of the formula

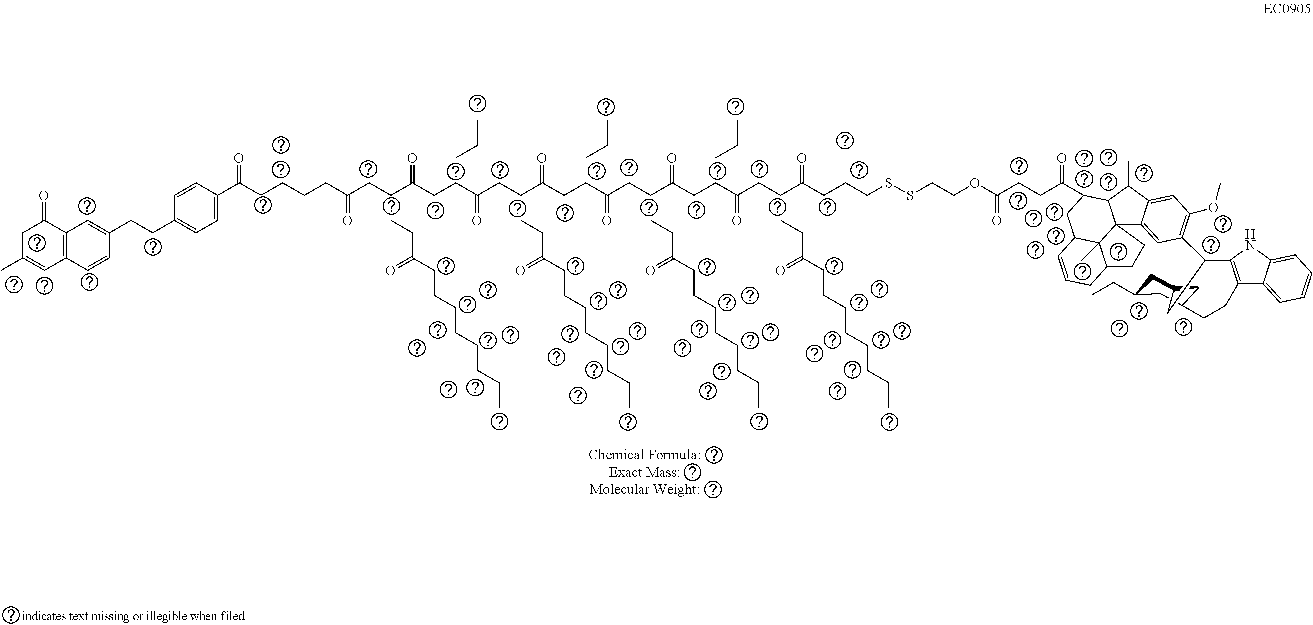

for treating urinary bladder cancer is disclosed. In one example embodiment, the urinary bladder cancer treated is invasive transitional cell carcinoma (InvTCC).

In one example embodiment, EC0905 is used in a composition, and use of the composition further comprises a pharmaceutically acceptable carrier. In one example embodiment, the use of the pharmaceutically acceptable carrier comprises a liquid. In some embodiments, the liquid carrier used is saline, glucose, an alcohol, a glycol, an ester, an amide, or a combination thereof.

In other embodiments, the use of the composition is an inhalation dosage form, an oral dosage form, or a parenteral dosage form. In some example embodiments, the parenteral dosage form used is an intradermal dosage form, a subcutaneous dosage form, an intramuscular dosage form, an intraperitoneal dosage form, an intravenous dosage form, or an intrathecal dosage form.

In another example embodiment, the use of the compound or composition is in the form of a solid. In one example embodiment, the use of the compound or composition is in the form of a suspension. In some embodiments, the purity of the compound is at least 90%, 95%, 98%, or 99% based on weight percent.

In one example embodiment, an immunohistochemical method for detecting folate receptors in urinary bladder cancer cells is disclosed, the method comprising the steps of contacting the urinary bladder cancer cells with an antibody having binding specificity for a folate receptor, and detecting folate receptor expression on the urinary bladder cancer cells, wherein the urinary bladder cancer cells are invasive transitional cell carcinoma cells (InvTCC).

In one example embodiment, the antibody is a polyclonal antibody.

In one example embodiment, the antibody is a monoclonal antibody.

In one example embodiment, the antibody has binding specificity for folate receptor-α.

### BRIEF DESCRIPTION OF THE DRAWINGS

FIG. 1 shows the structure of the EC0905 conjugate.

FIG. 2 shows PU17 IHC staining in canine kidney (Left Panels: negative control; Right Panels: positive control).

FIG. 3 shows PU17 IHC staining in canine invasive urinary bladder cancer (transitional cell carcinoma. InvTCC) (Left Panel: Canine InvTCC positive immunoreactivity with PU17; Right Panel: negative control).

FIG. 4 shows canine InvTCC IHC stain intensity in the cell membrane and cytoplasm.

FIG. 5 shows canine InvTCC metastases in the lung (top right), lymph node (bottom left), and kidney (bottom right) using PU17 IHC.

FIG. 6 shows PU17 IHC staining in normal canine bladder.

FIG. 7 shows the uptake of EC20 in urethral mass in dogs with InvTCC following shielding of the bladder and liver.

FIG. 8 shows EC20 scanning in a dog with InvTCC.

FIG. 9 shows EC20 scanning and thoracic radiograph in a dog with InvTCC.

FIG. 10 shows EC20 scanning and thoracic radiograph in a dog with InvTCC.

FIG. 11 shows EC20 scanning in a dog with InvTCC.

FIG. 12 shows EC20 scanning in a dog with InvTCC.

FIG. 13 shows bladder mapping with ultrasonography before (left panels) and after (right panels) EC0905 treatment in a dog.

FIG. 14 shows PU17 IHC staining in human kidney controls (Left panel: negative control; Right panel: positive control).

FIG. 15 shows PU17 IHC staining in human InvTCC.

FIG. 16 shows PU17 IHC staining in human InvTCC.

FIG. 17 shows PU17 IHC staining in human InvTCC.

FIG. 18 shows the results of folate receptor binding assays and PU17 IHC staining for human InvTCC samples (n=17).

### DETAILED DESCRIPTION OF ILLUSTRATIVE EMBODIMENTS

A method of treatment of a cancer is disclosed, the method comprising administering to a patient having cancer a therapeutically effective amount of a compound of the formula:

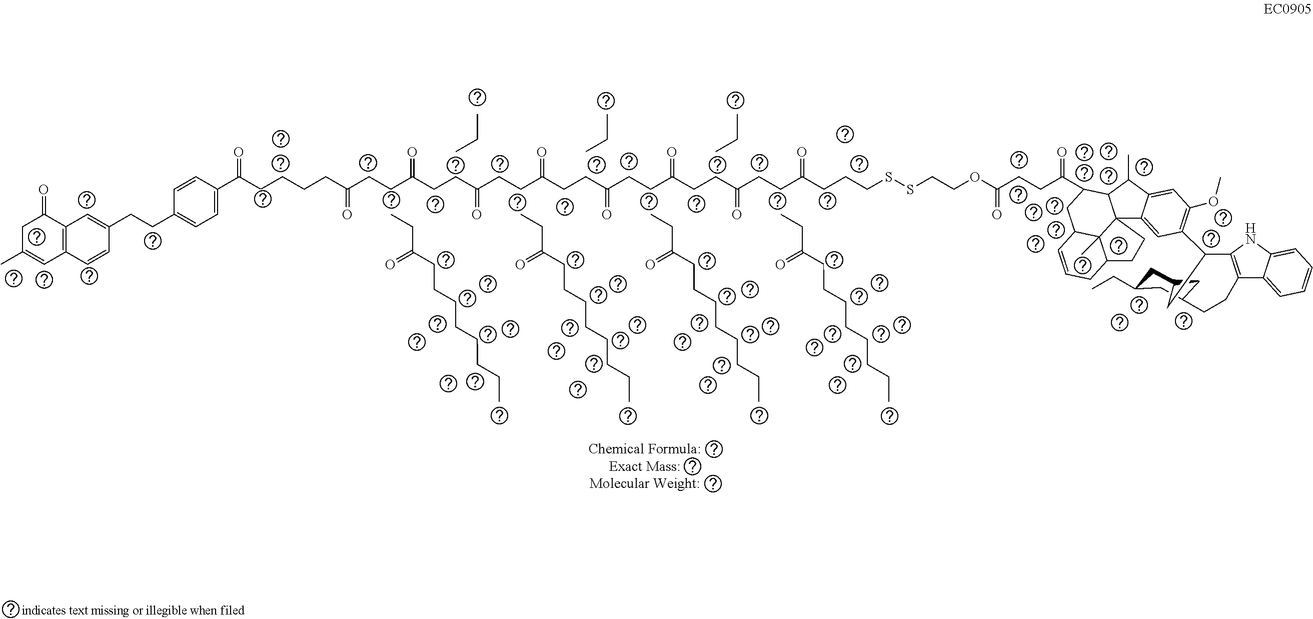

In some example embodiments, the cancer is urinary bladder cancer. Non-limiting examples of urinary bladder cancer include invasive transitional cell carcinoma (InvTCC) and low grade superficial urinary bladder cancer.

In some example embodiments, the compound is in a composition and the composition further comprises a pharmaceutically acceptable carrier. The compound or the composition may be an inhalation dosage form, an oral dosage form, or a parenteral dosage form. In currently preferred embodiments, the compound or the composition is in a parenteral dosage form. The parenteral dosage form may be an intradermal dosage form, a subcutaneous dosage form, an intramuscular dosage form, an intraperitoneal dosage form, an intravenous dosage form, or an intrathecal dosage form.

In some example embodiments, the pharmaceutically acceptable carrier comprises a liquid. The liquid carrier is saline, glucose, an alcohol, a glycol, an ester, an amide, or a combination thereof. In some example embodiments, the compound or the composition is in the form of a solid. Alternatively, the compound or the composition may be in the form of a suspension. The purity of the compound may be at least 90, 95, 98, or 99%, or more, based on weight percent. In some example embodiments, the compound is a pharmaceutically acceptable salt of EC0905. In certain embodiments, the pharmaceutically acceptable salt of EC0905 is a sodium salt.

Other embodiments include use of a compound of the formula:

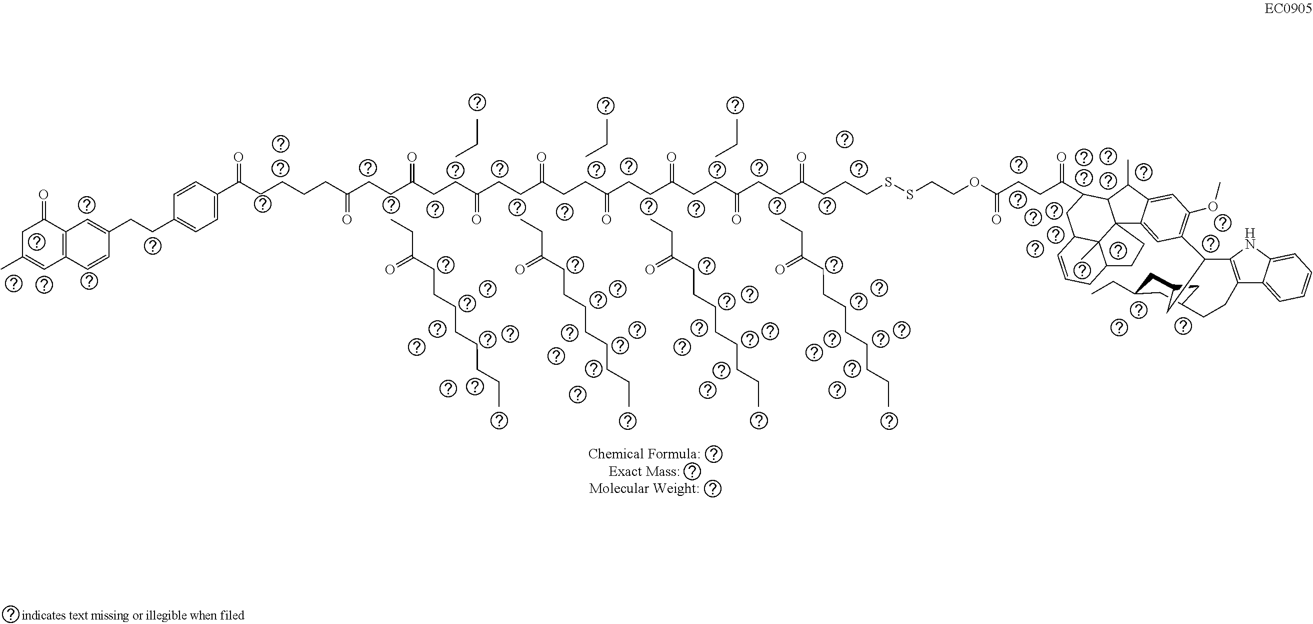

for treating urinary bladder cancer. In some example embodiments, the urinary bladder cancer treated by the used compound is invasive transitional cell carcinoma (InvTCC) or low grade superficial urinary bladder cancer.

In some example embodiments, the compound used to treat the urinary bladder cancer is in the form of a composition. In some example embodiments, the composition further comprises a pharmaceutically acceptable carrier. In some example embodiments, the compound or the composition is an inhalation dosage form, an oral dosage form, or a parenteral dosage form. In preferred embodiments, the compound or the composition used to treat the urinary bladder cancer is a parenteral dosage form, and the parenteral dosage form is an intradermal dosage form, a subcutaneous dosage form, an intramuscular dosage form, an intraperitoneal dosage form, an intravenous dosage form, or an intrathecal dosage form.

In some example embodiments, the pharmaceutically acceptable carrier comprises a liquid. The liquid carrier is saline, glucose, an alcohol, a glycol, an ester, an amide, or a combination thereof.

In some example embodiments, the compound or the composition used to treat the urinary bladder cancer is in the form of a solid. Alternatively, the compound or the composition may be in the form of a suspension. In some example embodiments, the purity of the compound used to treat the urinary bladder cancer is at least 90% based on weight percent. Alternatively, the purity of the compound used to treat the urinary bladder cancer may at least 95%, 98° i®, or 99%, or more, based on weight percent. In some example embodiments, the compound used is a pharmaceutically acceptable salt of EC0905. In certain embodiments, the pharmaceutically acceptable salt of EC0905 is a sodium salt.

An immunohistochemical method for detecting folate receptors in urinary bladder cancer cells is disclosed. The method comprises the steps of contacting the urinary bladder cancer cells with an antibody directed to a folate receptor, and detecting folate receptor expression on the urinary bladder cancer cells, wherein the urinary bladder cancer cells are invasive transitional cell carcinoma cells (InvTCC). In some example embodiments, the antibody is a polyclonal antibody. Alternatively, the antibody may be a monoclonal antibody. In some example embodiments, the antibody is directed to folate receptor-α.

In any of the various embodiments described herein, the following features may be present where applicable, providing additional embodiments of the invention. For all of the embodiments, any applicable combination of embodiments is also contemplated.

The methods described herein can be used for both human clinical medicine and animals. Thus, the patient treated using the methods herein described can be human or can be a laboratory, agricultural, domestic, or wild animal. Thus, the methods described herein are useful for treating humans, laboratory animals such rodents (e.g., mice, rats, hamsters, etc.), rabbits, monkeys, chimpanzees, domestic animals (e.g., dogs and cats), agricultural animals such as cows, horses, pigs, sheep, goats, ostriches, and wild animals in captivity such as bears, pandas, lions, tigers, leopards, elephants, zebras, giraffes, gorillas, dolphins, sea lions, or whales.

In other embodiments of the methods and uses described herein, pharmaceutically acceptable salts of the conjugate described herein can be used. Pharmaceutically acceptable salts of the conjugate described herein include the acid addition salts or salts made with bases.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate or trifluoroacetate salts.

Suitable salts made with bases of the conjugate described herein for use in the method of treatment are formed from bases which form non-toxic salts. Illustrative examples include the arginine, benzathine, calcium, choline, diethylamine, diolamine, glycine, lysine, magnesium, meglumine, olamine, potassium, sodium, tromethamine or zinc salts. Hemi-salts of acids and bases may also be formed, for example, hemi sulphate and hemi-calcium salts.

In one embodiment, the conjugate described herein may be administered in the method of treatment as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Methods for the preparation of pharmaceutical compositions suitable for the delivery or administration of the conjugate or additional chemotherapeutic agents to be administered with the conjugate will be readily apparent to those skilled in the art. Such methods for their preparation may be found, for example, in Remington: The Science & Practice of Pharmacy, 21st Edition (Lippincott Williams & Wilkins, 2005), incorporated herein by reference.

In one embodiment, a pharmaceutically acceptable carrier for delivery of the conjugate for se in the method of treatment may be selected from any and all solvents, dispersion media, coatings, antibacterial and antifungal agents, isotonic and absorption delaying agents, and the like, or combinations thereof, that are physiologically compatible. In some embodiments, the carrier is suitable for parenteral administration. Pharmaceutically acceptable carriers include sterile aqueous solutions or dispersions and sterile powders for the preparation of sterile injectable solutions or dispersions.

In various embodiments, liquid formulations for use in the method of treatment may include suspensions and solutions. Such formulations may comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid.

In one embodiment, an aqueous suspension may contain the active materials in admixture with appropriate excipients for use in delivery of the conjugate for the method of treatment described herein. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions for use in the method of treatment may also contain one or more preservatives, for example, ascorbic acid, ethyl, n-propyl, or p-hydroxybenzoate; or one or more coloring agents.

In one aspect, the conjugate may be administered for the method of use directly into the blood stream, into muscle, or into an internal organ. Suitable routes for such parenteral administration include intravenous, intraarterial, intraperitoneal, inhalation, intrathecal, epidural, intracerebroventricular, intraurethral, intrasternal, intracranial, intratumoral, intramuscular, or subcutaneous delivery. Suitable means for parenteral administration include needle (including microneedle) injectors, needle-free injectors or infusion techniques.

Examples of parenteral dosage forms for use in the method of treatment include aqueous solutions of the active agent, in an isotonic saline, glucose (e.g., 5% glucose solutions), or other well-known pharmaceutically acceptable liquid carriers such as liquid alcohols, glycols, esters, and amides. In one aspect of the present embodiment for the method of treatment, any of a number of prolonged release dosage forms known in the art can be administered such as, for example, by using biodegradable carbohydrate matrices, or a slow pump (e.g., an osmotic pump).

In one illustrative aspect, parenteral formulations for use in the method of treatment are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments of the method of treatment described herein, any of the liquid formulations described herein may be adapted for parenteral administration of the conjugates. The preparation of parenteral formulations under sterile conditions, for example, by lyophilization under sterile conditions, may readily be accomplished using standard pharmaceutical techniques well-known to those skilled in the art.

Any effective regimen for administering EC0905 can be used. For example, EC0905 can be administered as a single dose, or can be divided and administered as a multiple-dose daily regimen. Further, a staggered regimen, for example, one to five days per week can be used as an alternative to daily treatment, and for the purpose of the methods and uses described herein, such intermittent or staggered daily regimen is considered to be equivalent to every day treatment and is contemplated. In one illustrative embodiment the patient is treated with multiple injections of EC0905 to eliminate the tumor(s). In one embodiment, the patient is injected multiple times (preferably about 2 up to about 50 times) with EC0905, for example, at 12-72 hour intervals or at 48-72 hour intervals. Additional injections of EC0905 can be administered to the patient at an interval of days or months after the initial injection(s) and the additional injections can prevent recurrence of the cancer.

The unitary daily dosage of EC0905 can vary significantly depending on the patient condition, the disease state being treated, the purity of the compounds and their route of administration and tissue distribution, and the possibility of co-usage of other therapeutic treatments, such as radiation therapy. The effective amount to be administered to a patient is based on body surface area, mass, and physician assessment of patient condition. Effective doses can range, for example, from about 1 ng/kg to about 1 mg/kg, from about 1 μg/kg to about 500 μg/kg, and from about 1 μg/kg to about 100 μg/kg. These doses are based on an average patient weight of about 70 kg, and the kg are kg of patient body weight (mass).

In one embodiment, the EC0905 conjugate can be administered in a dose of from about 1.0 ng/kg to about 1000 μg/kg, from about 10 ng/kg to about 1000 μg/kg, from about 50 ng/kg to about 1000 μg/kg, from about 100 ng/kg to about 1000 μg/kg, from about 500 ng/kg to about 1000 μg/kg, from about 1 ng/kg to about 500 μg/kg, from about 1 ng/kg to about 100 μg/kg, from about 1 μg/kg to about 50 μg/kg, from about 1 μg/kg to about 10 μg/kg, from about 5 μg/kg to about 500 μg/kg, from about 10 μg/kg to about 100 μg/kg, from about 20 μg/kg to about 200 μg/kg, from about 10 μg/kg to about 500 μg/kg, or from about 50 μg/kg to about 500 μg/kg. The total dose may be administered in single or divided doses and may, at the physician's discretion, fall outside of the typical range given herein. These dosages are based on an average patient weight of about 70 kg and the “kg” are kilograms of patient body weight. The physician will readily be able to determine doses for subjects whose weight falls outside this range, such as infants and the elderly.

In another embodiment, EC0905 can be administered in a dose of from about 1 μg/m² to about 500 mg/m², from about 1 μg/m² to about 300 mg/m², or from about 100 μg/m² to about 200 mg/m². In other embodiments, EC0905 can be administered in a dose of from about 1 mg/m² to about 500 mg/m², from about 1 mg/m² to about 300 mg/m², from about 1 mg/m² to about 200 mg/m², from about 1 mg/m² to about 100 mg/m², from about 1 mg/m² to about 50 mg/m², or from about 1 mg/m² to about 600 mg/The total dose may be administered in single or divided doses and may, at the physician's discretion, fall outside of the typical range given herein. These dosages are based on m² of body surface area.

In another embodiment, compositions and/or dosage forms for administration of EC0905 for the method of treatment described herein are prepared from compounds with a purity of at least about 90%, or about 95%, or about 96%, or about 97%, or about 98%, or about 99%, or about 99.5%, or more. In another embodiment, compositions and or dosage forms for administration of EC0905 are prepared from compounds with a purity of at least 90%, or 95%, or 96%, or 97%, or 98%, or 99%, or 99.5%, or more.

For purposes of this specification and appended claims, unless otherwise indicated, all numbers expressing quantities, percentages or proportions, and other numerical values used in the specification and claims, are understood as being modified in all instances by the term “about.” Accordingly, unless indicated to the contrary, the numerical parameters set forth in the following specification and attached claims are approximations that may vary depending upon the desired properties sought to be obtained by the present invention.

In another embodiment, the methods and uses described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### EXAMPLES

### Example 1

### EC0905

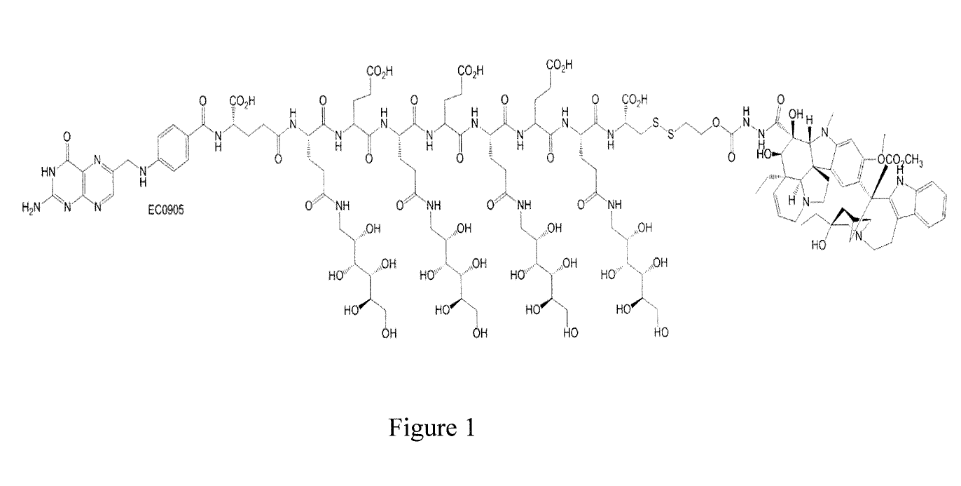

The structure of EC0905 is shown in FIG. 1. The carbohydrate-containing folate -spacer unit contains alternately repeating acidic (Glu) and saccharo-amino acids, thus providing high water-solubility of the final drug conjugate under physiological conditions (Vlahov et al., J. Org. Chem., 2010, 75, 3685-3691). This unit is assembled using standard fluorenylmethyloxycarbonyl-based solid phase peptide synthesis (Fmoc SPPS) on a Wang-resin. Desacetylvinblastine Hydrazide (DA VLBH) was prepared from commercially available vinblastine (VLB) sulfate (Barnett et al., J. Med. Chem., 1978, 21, 88). Details regarding the preparation of EC0905 are provided below in Examples 2 and 3. As shown in Example 3, an activated carbonate (3) (Vlahov et al., Bioorg. & Medicinal Chem. Lett., 2006, 16, 5093) served as a heterobifunctional crosslinker to provide the drug-linker intermediate (4) for use in the assembly of the final conjugate. Treatment of a solution of folate -spacer in H₂O under Argon and under extensive stirring with the Drug-Linker (4) unit resulted in a yellow suspension. According to the HPLC profile, the reaction was completed in 15 minutes. HPLC purification gave pure conjugate EC0905.

### Example 2

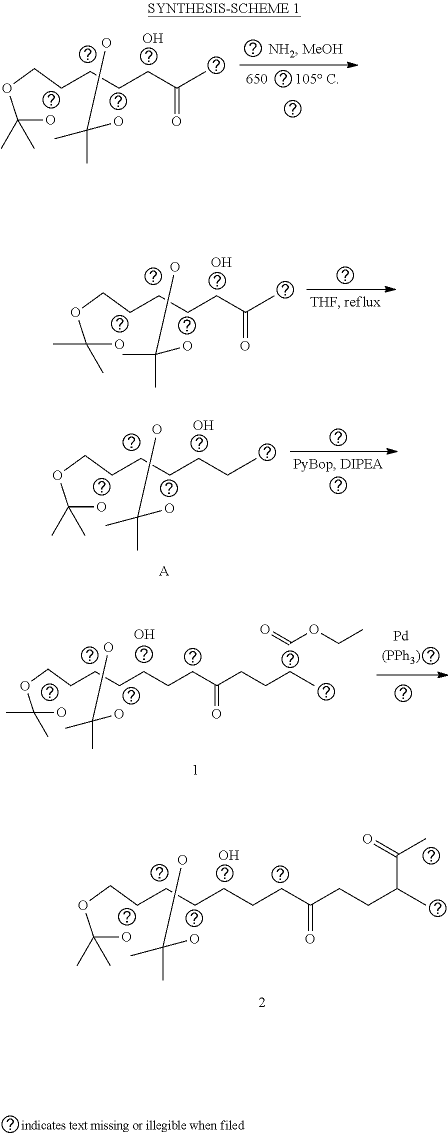

### Synthesis of 3,4;5,6-di-O-isopropylidene-1-amino-1-deoxy-(Fmoc-Glu-Oallyl)-D-glucitol (1)

Fmoc-Glu-OAll (2.17 g, 1 eq), PyBOP (2.88 g, 1 eq), and DIPEA (1.83 mL, 2 eq) were added to a solution of 3,4;5,6-di-O-isopropylidene-1-amino-1-deoxy-D-glucito (A) (1.40 g, 5.3 mmol) in dry DMF (6 mL) and the reaction mixture was stirred at room temperature under Ar for 2 h, The solution was diluted with EtOAc (50 mL), washed with brine (10 mL×3), the organic layer separated, dried (MgSO₄), filtered, and concentrated to give a residue, which was purified by a flash column (silica gel, 60% EtOAc/petroleum ether) to afford the title compound (1.72 g, 50%) as a solid.

### Synthesis of 3,4;5,6-di-O-isopropylidene-1-amino-1-deoxy-(Fmoc-Glu-OH)-D-glucitol (2)

Pd(Ph₃)₄ (300 mg, 0.1 eq) was added to a solution of (1) (1.72 g, 2.81 mmol) in NMM/AcOH/CHCl₃ (2 mL/4 mL/74 The resulting yellow solution was stirred at room temperature under Ar for 1 h, to which was added a second portion of Pd(Ph₃)₄(300 mg, 0.1 eq). After stirring for an additional 1 h, the reaction mixture was washed with 1 N HCl (50 mL×3) and brine (50 mL), organic layer separated, dried (MgSO₄), filtered, and concentrated to give a yellow foamy solid, which was subject to chromatography (silica gel, 1% MeOH/CHCl₃ followed by 3.5% MeOH/CHCl₃) to give (2) (1.3 g, 81%) as a solid. Compound (A) may be obtained as outlined in the scheme and as described in WO 2009/002993 at pages 68 and 81-82.

### Synthesis of the Folate-Spacer Pte-γGlu-(Glu(1-amino-1-deoxy-D-glucitol)-Glu)₃-Glu(1-amino-1-deoxy-D-glucitol)-Cys-OH

H-Cys(4-methoxytrityl)-2-chlorotrityl-resin (0.17 g, 0.10 mmol) was loaded into a peptide synthesis vessel and washed with i-PrOH (3×10 mL), followed by DMF (3×10 mL). To the vessel was introduced a solution of 3,4;5,6-di-O-isopropylidene-1-amino-1-deoxy-(Fmoc-Glu-OH)-D-glucitol (0.13 mmol) in DMF, i-PrNEt (2 eq.), and PyBOP (1 eq.). The resulting solution was bubbled with Ar for 1 hr, the coupling solution was drained, and the resin washed with DMF (3×10 mL) and i-PrOH (3×10 mL). Kaiser tests were performed to assess reaction completion. Fmoc deprotection was carried out using 20% piperidine in DMF (3×10 mL). This procedure was repeated to complete all coupling steps (1.9 eq. of Fmoc-Glu(Ot-Bu)-OH and Fmoc-Glu-Ot-Bu, and 1.6 eq. of N¹⁰TFA-pteroic acid were used on each of their respective coupling steps). After the pteroic acid coupling, the resin was washed with 2% hydrazine in DMF (3× for 5 min. each) lo remove the trifluoroacetyl protecting group. The resin was washed with DMF (3×10 mL) and MeOH (10 mL) and dried under reduced pressure. The peptide was cleaved from the resin in the peptide synthesis vessel using a cleavage mixture consisting of 92.5% CF₃CO₂H, 2.5% H₂O, 2.5% triisopropylsilane, and 2.5% ethanedithiol. 25 mL of the cleavage mixture was added to the peptide synthesis vessel and the reaction was bubbled under Ar for 10 min. The resin was treated with two additional 15 mL quantities of the cleavage mixture for 5 minutes each. The cleavage mixture was concentrated to ca. 5 mL and ethyl ether was added to induce precipitation The precipitate was collected by centrifugation, washed with ethyl ether 3 times, and dried under high vacuum, resulting in the recovery of ca.100 mg of crude material. The compound was purified by prep. HPLC (mobile phase: A=10 mM ammonium acetate pH=5, B=ACN; method: 0% B to 20% B in 25 minutes at 15 mL/min). The pure fractions were pooled and freeze-dried, furnishing folate-spacer unit (51%).

### Example 3

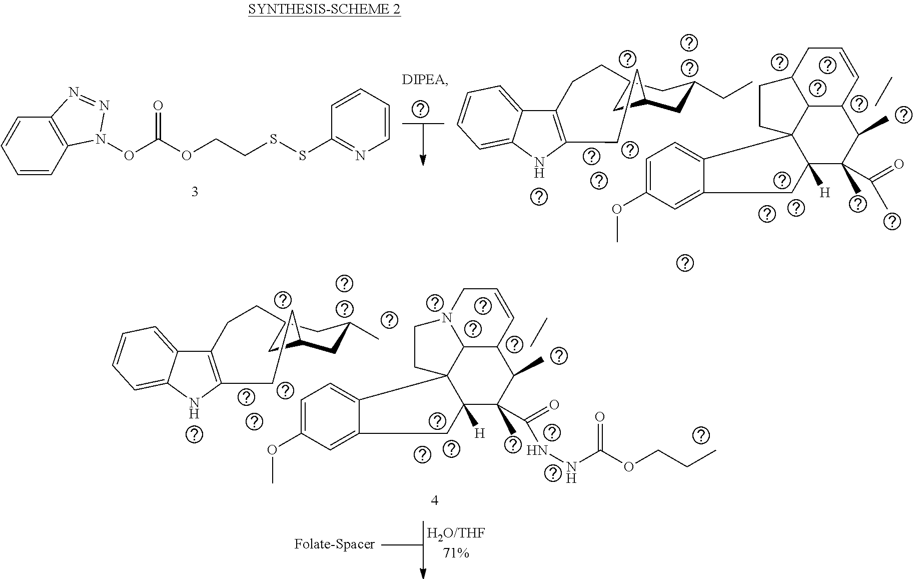

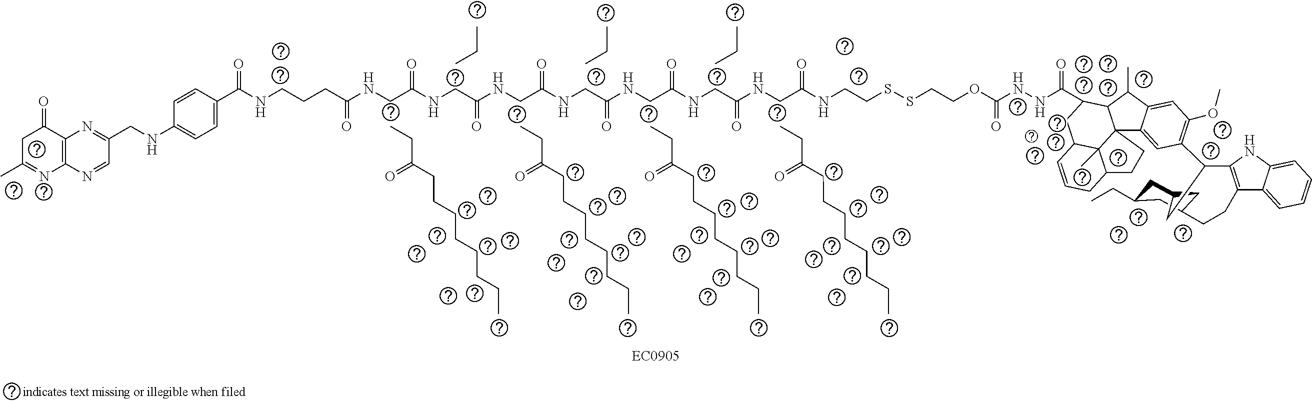

### Synthesis of EC905: Pte-γGlu-(Glu(1-amino-1-deoxy-D-glucitol)-Glu)₃-Glu(1-amino-1-deoxy-D-glucitol)-Cys(S-ethyl-3-(4-desacetylvinblastinyl)hydrazinecarboxylate)

In a polypropylene centrifuge bottle, the folate-spacer (0.015 mmol) was dissolved in 2.5 mL of Ar sparged water. In another flask, a saturated NaHCO₃ solution was Ar sparged for 10 min. The pH of the linker solution was carefully adjusted, with argon bubbling, to 6.9 using the NaHCO₃ solution. Vinblastine hydrazide-linker (4) (15 mg, 1.0 eq) in 2.5 mL of tetrahydrofuran (THF) was added quickly to the above solution. The resulting clear solution was stirred under argon. Progress of the reaction was monitored by analytical HPLC (2 mM sodium phosphate buffer, pH=7.0 and acetonitrile). After 20 min, 2 mM phosphate buffer (pH=7, 12 mL) was added to the reaction. The resulting cloudy solution was filtered and the filtrate was injected on the prep-HPLC (mobile phase: A=2 mM sodium phosphate pH=7, B=ACN; method: 1% B to 50% B in 25 minutes at 26 mL/min). Pure fractions were pooled and freeze-dried resulting in the recovery of EC0905 as a fluffy yellow powder (71%).

### Example 4

### Tissues

Formalin fixed (for IHC) and snap frozen (for folate binding assay) sections from human InvTCC, lymph node metastases, and normal bladder were obtained from the Indiana University Simon Cancer Center Tissue Bank, Indianapolis, Ind. Specimens of canine InvTCC (primary, lymph node and lung metastases) were obtained from the Indiana Animal Disease Diagnostic Laboratory and from the Purdue Comparative Oncology Program, Purdue University School of Veterinary Medicine, West Lafayette, Ind.

### Example 5

### Immunohistochemistry

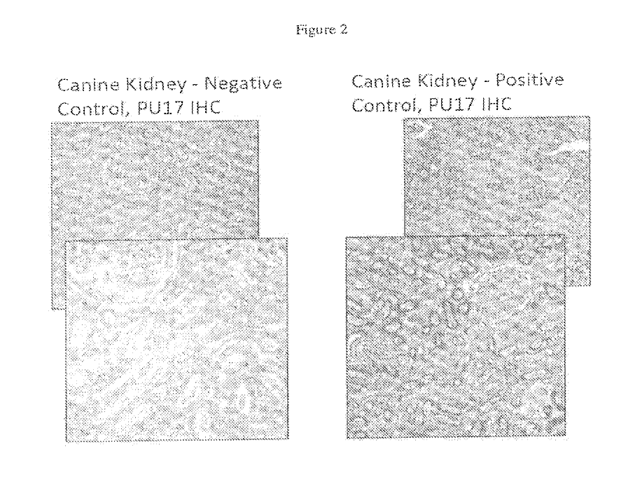

Analysis of FR expression in human and canine InvTCC, with comparison to normal bladder tissues, was performed using immunohistochemistry (IHC). Briefly, 5 μm sections were cut from paraffin-embedded human and canine InvTCC tissues and placed on Superfrost® slides. Sections were dewaxed in xylene and rehydrated in descending percentages of alcohol. Target Retrieval solution (Dako Corp., Carpinteria, Calif.) was used according to the manufacturer's instructions. The sections were immersed in 3% hydrogen peroxide to block the endogenous peroxidase, and then blocked with SNIPER (MACH3 detection, Biocare Medical, Walnut Creek, Calif.). This was followed by incubation with primary antibody for 2 hrs at room temperature (i.e., a rabbit polyclonal antibody, PU17). To determine the appropriate antibody for canine tissues, experiments were performed in which several different antibodies were employed, and detection of the apical expression of FR in proximal renal tubular epithelial cells was used as a positive control (FIG. 2). There was consistent and specific immunoreactivity to PU17 with the positive control cells, and thus this antibody was used for canine tissues. In the studies, paired slides were stained using Universal Negative control serum (Biocare) (FIG. 2). FR immunoreactive complexes were detected using MACH4 Universal HRP-Polymer for canine InvTCC slides and MACH4 mouse probe and Universal HRP polymer for human InvTCC slides. Immunoreactive complexes were visualized using DAB substrate (Vector Laboratories, Inc., Burlingame, Calif.). Slides were counter stained in hematoxylin-1 (Richard-Allan Scientific, Kalamazoo, Mich.) and cover-slipped in 50:50 xylene/permount (Fisher Scientific).

Slides were reviewed independently by two investigators. Any discrepancy in the assessment of the two reviewers was resolved by screening those cases concurrently to reach a consensus, The percent of positively immunostained tumor cells was categorized as 0 to 3 as follows: 0=<10% of cells, 1=10-20% of cells, 2=21-50% of cells, and 3=51-100% of cells staining positively. The intensity of immunostaining was grades on a scale of 0-3 where 0=no staining, 1=equivocal staining, 2=moderate to intense staining, and 3=highest intensity staining. Tissue samples were considered positive for FR expression if immunoreactivity was noted in 2:10% of the tumor cells.

### Canine

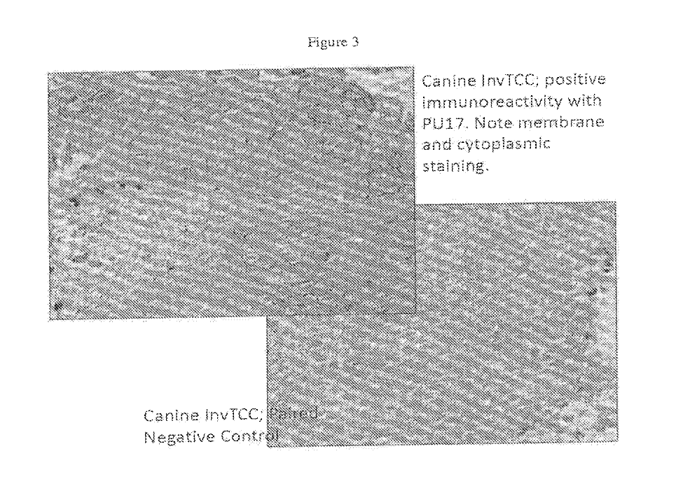

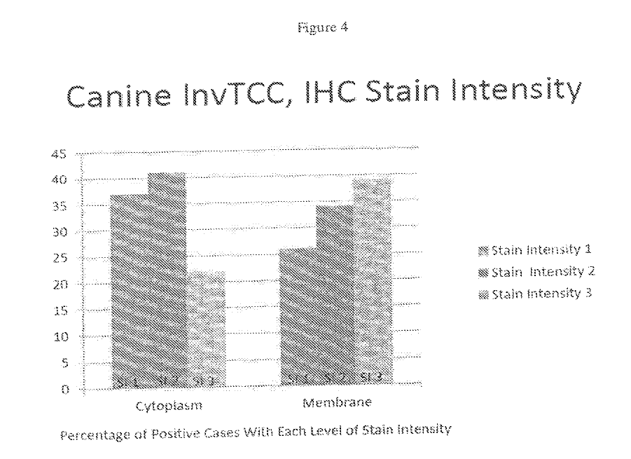

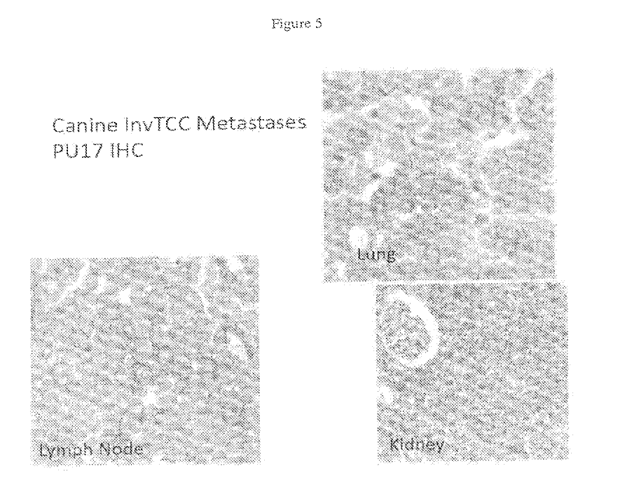

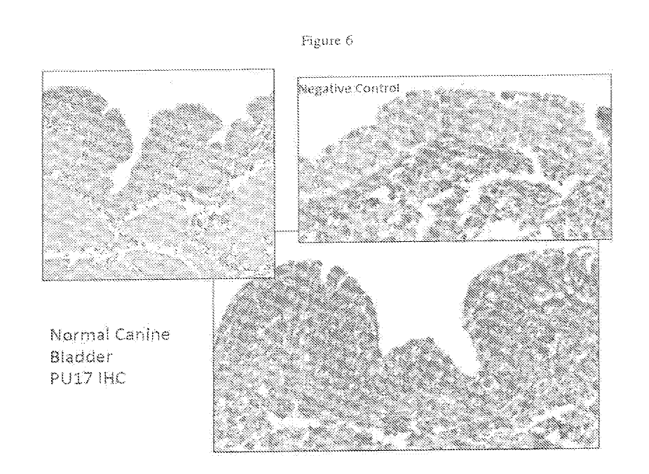

Samples from 74 dogs were studied. IHC staining of canine InvTCC using PU17 is shown in FIG. 3. All dogs had intermediate to high grade InvTCC. The median age was 11 years (range 4-17), There were 40 spayed female, 32 neutered male, and 2 intact male dogs. A variety of dog breeds were represented. Nodal metastases were present in 23 dogs (31%), and distant metastases were present in 28 dogs (38%). FR expression was detected in 56 of 74 (76%) primary tumors, in 7 of 12 (58%) nodal metastases, and in 10 of 21 (48%) of lung metastases with staining intensity of 2-3+ in the majority of cases (Table 1). In 67% of cases the FR expression in the primary tumor and lung metastases were similar (either positive in both sites, or negative in both sites); in 33% of cases FR expression was detected in the primary tumor but not in metastases. IHC stain intensity for canine InvTCC samples is shown in FIG. 4. Folate receptor expression in canine InvTCC metastases for lung, lymph node, and kidney are shown in FIG. 5. Immunoreactivity was noted in the epithelial cells in 8 of 8 normal bladders from dogs (FIG. 6) (≧80% cells positive, 2-3+ staining intensity, typically membrane and cytoplasmic).

### Human

Immunohistochemistry was performed on tumor samples from 37 humans with InvTCC. Nodal metastases were present in at least 23 of the patients. Distant metastases were not reported as samples were collected from patients undergoing cystectomy, and cystectomy is usually reserved for patients without distant metastases. The median patient age was 64.5 years (range 39-82 years), and there were 25 male and 11 female patients (gender not recorded in one patient).

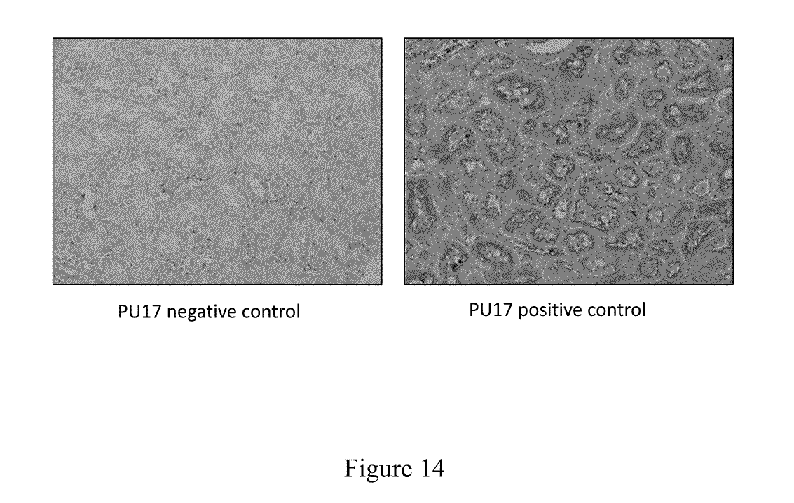

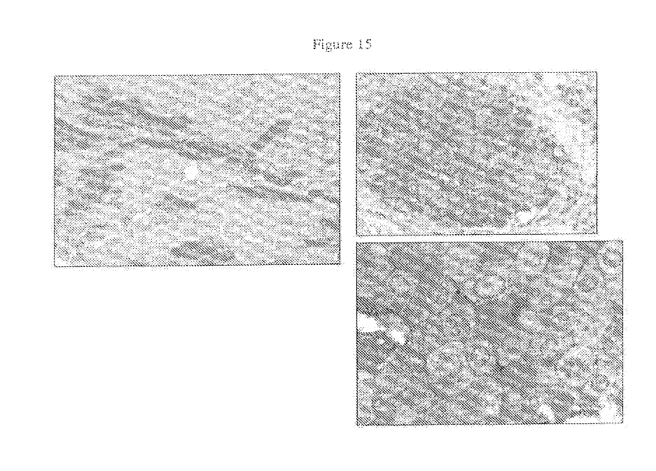

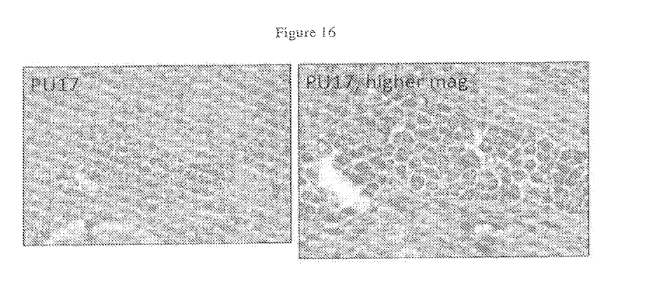

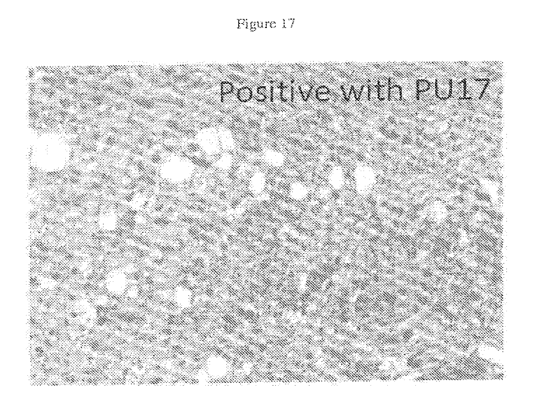

Immunoreactivity to PU17 was noted in tumor cells in 29 of 37 (78%) primary tumors and in 12 of 15 (80%) nodal metastases (Table I). Immunoreactivity was noted in >50% of tumor cells in most sections, and staining intensity was usually 1-2+. FIG. 14 shows negative and positive controls for IHC staining using PU17 in human kidney samples. FIGS. 15, 16, and 17 show PU17 HC staining in human InvTCC. The epithelium adjacent to the tumor was studied in 5 cases, and immunoreactivity to PU17 was noted in epithelial cells in all 5 cases (60% positive cells, 2-3 stain intensity, mostly cytoplasmic). Membrane staining intensity was 2-3+ and was typically present in 50% or less of the tumor cells. The epithelium adjacent to the tumor was negative in all 5 cases. FIG. 17 shows results of PU17 IHC staining for human InvTCC samples (n=1 7).

TABLE 1

| | |
|---|---|
| PU17 IHC in Dogs | PU17 IHC in Humans |

Primary Tumor

| Number | 74 | | 37 | |
|---|---|---|---|---|
| Number | 56 (76%) | | 29 (78%) | |
| positive | | | | |
| Location | | | | |
| Membrane | 6 (11%) | | 0 | |
| Cytoplasm | 18 (32%) | | 19 (66%) | |
| Both | 32 (57%) | | 10 (54%) | |
| % positive | | | | |
| cells | Membrane | Cyto | Membrane | Cyto |
| 10-19% | 9/38 (24%) | 3/50 (6%) | 1/10 (10%) | 2/29 (7%) |
| 20-49% | 8/38 (21%) | 6/50 (12%) | 0/10 | 5/29 (17%) |
| 50-79% | 12/38 (31%) | 14/50 (28%) | 7/10 (7%) | 15/29 (52%) |
| ≧80% | 9/38 (24%) | 27/50 (54%) | 2/10 (20%) | 7/29 (24%) |

Lymph Node Metastases

| | | |
|---|---|---|
| Number | 12 | 15 |
| Number | 7 (58%) | 12 (80%) |
| positive | | |

Lung Metastases

| | | |
|---|---|---|
| Number | 21 | Not available |
| Number | 10 (48%) | |
| positive | | |

### Example 6

### Folae Binding Assay

Folate binding assays were conducted using human and canine InvTCC samples, with comparison to normal bladder tissues. Measurement of folate binding in InvTCC and control tissues was accomplished using a previously described method with modification. Samples were loaded into the upper chambers of paired filtration tubes and diluted with a solubilizing solution. The filtration tubes were centrifuged, and the filters treated with acetate solution to remove endogenous folate followed by centrifugation and two washes with the solubilizing solution. Then pairs of filtration tubes were incubated either with a 1000X cold folate in a binding solution or with the binding solution alone, and samples were incubated 2 hours at room temperature and then centrifuged. Binding solution containing ³H-folate was then added to all samples followed by incubation overnight at 4° C. with gentle agitation. The filters were centrifuged and washed with PBS containing n-octyl-3-D-glucopyranoside to remove the unbound ³H-folate. Bound ³H-folate retained on the filters was removed with PBS containing Triton X-100 and transferred into a vial with liquid scintillation cocktail, and the activity was measured in a Beckman LS6000IC Scintillation Counter (Brea, Calif.). Specific binding was determined by subtracting the activity in the presence of excess cold folate from the activity of the same sample without folate competition.

Folate binding to TCC tissues was detected ex vivo in samples from 9 dogs studied. The binding ranged from 0.17 to 3.1 pmol FR/mg protein (median 1.4 pmol FR/mg protein). All 9 cases had positive immunoreactivity in tumor cells with IHC. No differences were observed in the IHC findings between samples with the lowest vs the highest folate binding.

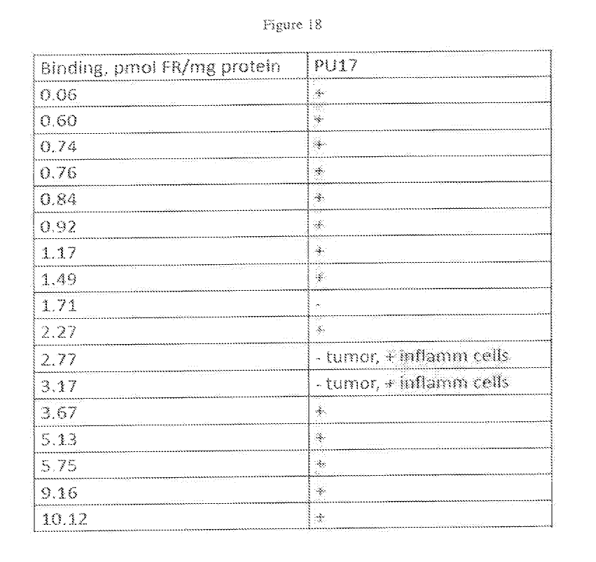

FIG. 18 shows results of folate receptor binding assays (FRβ) and PU17 IHC staining for human InvTCC samples (n=17).

### Example 7

### Scintigraphy

Nuclear scintigraphy was used to detect folate uptake in InvTCC in dogs. Following pet owner consent, privately-owned dogs with naturally-occurring InvTCC were imaged with a technetium-folate conjugate (^{99m}Tc-EC20). The conjugate was prepared as an individual dose for each dog. Briefly, 5 mCi ^{99m} Tc was added to EC20 solution (provided by Endocyte, Inc_{—} West Lafayette, IN), and the vial with the mixture was placed in a boiling water bath for 20 min. The ^{99m}Tc-EC20 was injected intravenously two hours prior to imaging. Dogs were placed under general anesthesia, and full body static images were acquired in right and left lateral, ventrodorsal, and dorsoventral recumbencies over a 90 second per view time period using a single head gamma camera (MiE Equine Scanner H.R. - Scintron VI, Elk Grove Village, Ill.) with a 60x39 cm detector, a 256×256 matrix, and low energy all purpose collimator. Various positional acquisitions were also obtained as dictated by tumor location. A board-certified veterinary radiologist interpreted all images acquired by nuclear scintigraphy. Following nuclear scintigraphy, all dogs were kept in hospital for ≧24 hours or until surface exposure rate was <30 mR/hr.

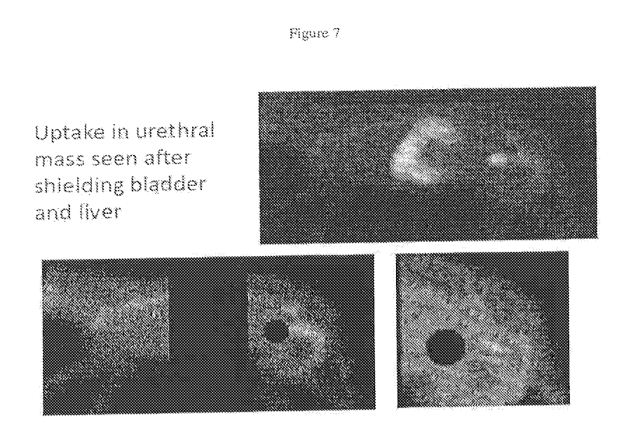

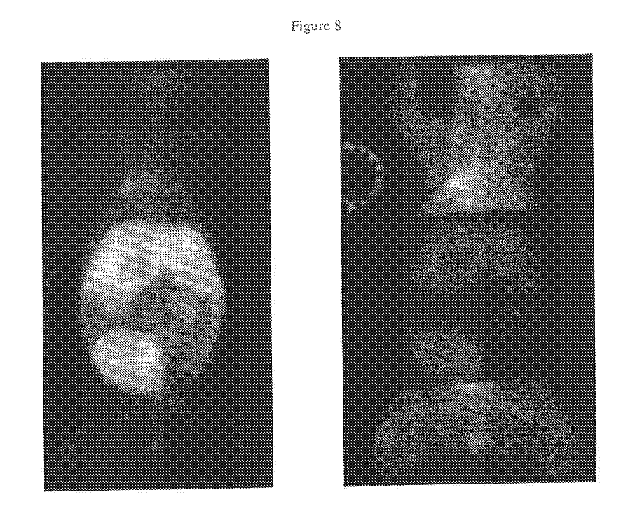

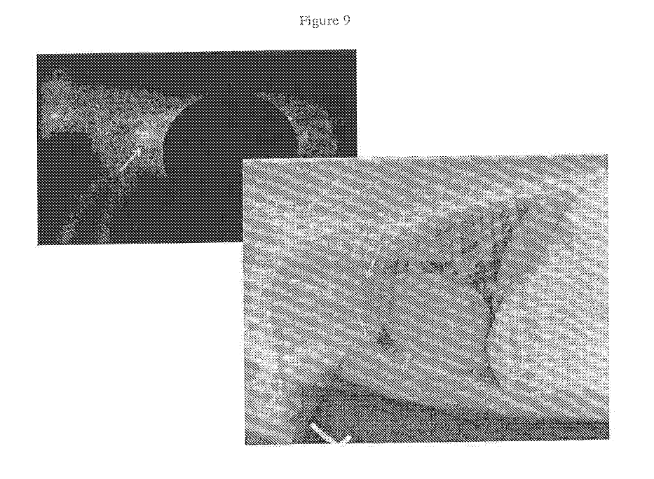

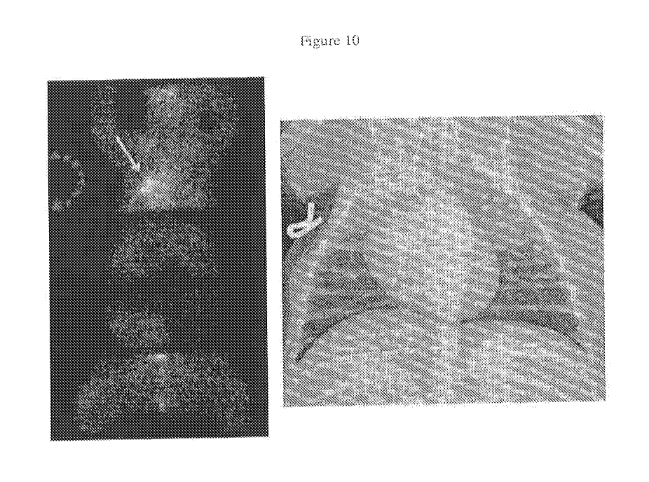

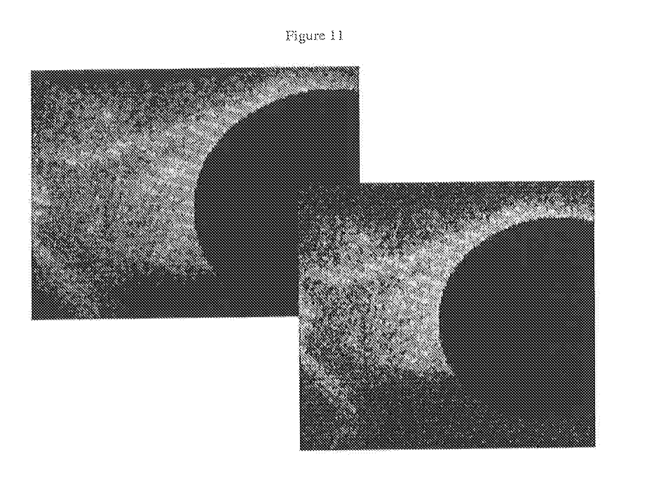

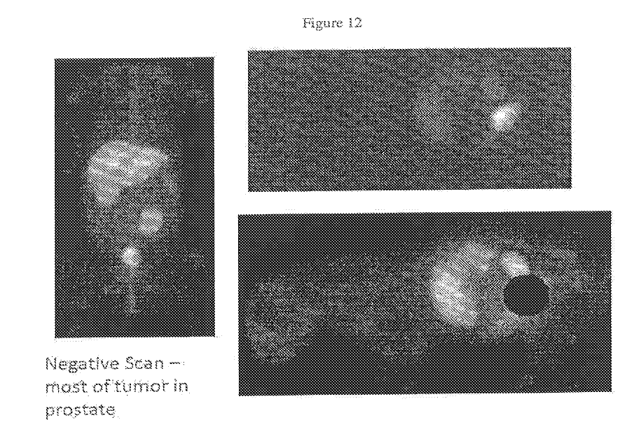

Scintigraphy with ^{99m}Tc-EC20 was performed in 13 dogs, with shipment of the ^{99m}Tc scheduled to allow scanning with approximately 5 mCi. The actual mean activity of the injected conjugate was 6.2 mCi/dog (range 3.8-10.2 mCi/dog). EC20 uptake was detected in the cancer (in primary and/or metastases) in 12 of the 13 dogs. The 12 dogs with positive scans also had FR expression in tumor cells detected by IHC. One dog with bulky spread of the cancer had a negative scan and negative IHC. With the EC20 being eliminated through the urine, special steps were required to observe uptake in the bladder masses. These included removing the urine from the bladder and distending the bladder with sterile saline, and individual dog positioning based on the location of the cancer within the bladder. Uptake in the cancer in the urethra and prostate could be observed when the bladder (and residual radioactivity) were shielded (FIG. 7). Six of the 13 dogs scanned had biopsy-confirmed distant metastases, and 2 dogs had radiographic evidence of lung metastases not confirmed by biopsy (FIGS. 8 to 12). EC20 uptake was observed in 4 of 6 dogs with biopsy-confirmed metastases, and in 2 of 2 dogs with radiographic evidence of metastases, In 2 of the dogs, the metastatic lesions were obscured by nonspecific EC20 uptake in the liver. Nonspecific uptake of the ^{99m}Tc-EC20 was noted in the liver of all dogs, as has been previously reported in humans. Shielding the radioactivity in the liver was usually necessary to observe the radioactivity in lung metastases, although lesions very caudal in the lung field were still not observed.

### Example 8

### Study of Folate-Vinblastine Conjugate in Dogs with INVTCC

A study was performed in dogs with naturally-occurring InvTCC to investigate antitumor activity and toxicity of folate targeted vinblastine treatment. The study of folate targeted vinblastine was performed in privately owned dogs with naturally-occurring InvTCC at the Purdue University Veterinary Teaching Hospital (PUVTH), Inclusion criteria for dogs in the study included: measurable, histologically-confirmed InvTCC; positive folate uptake detected by scintigraphy or FR expression observed in the tumor via IHC; expected survival of at least 6 weeks; and written dog owner consent. The study was open to dogs that had failed other therapies or who were not eligible for standard therapy.

The folate-vinblastine conjugate EC0905 was used. Briefly, EC0905 is a water-soluble folate conjugate of desacetylvinblastine monohydrazide (DA VLBH), which is constructed with the DA VLBH drug moiety attached to a hydrophilic folate-peptide compound via an endosome-cleavable disulfide bond (FIG. 1). A dose escalation study was performed. EC0905 was administered IV once weekly (starting dose 0.2 mg/kg), Dose escalation was performed within and between dogs with at least 3 dogs in each dose group, and at least 6 dogs treated at the maximum tolerated dose (MTD) (increased by 0.02 mg/kg in each dose group). Toxicity was assessed by CBCs, serum chemistry profiles, urinalyses, physical exams, and owner observations. Toxicity was classified by Veterinary Cooperative Oncology Group (VCOG) criteria. The MTD was defined as the highest dose that resulted in 0 of 6 dogs having grade 4 toxicity and 0 or 1 of 6 dogs having grade 3 toxicity.

The EC0905 dose was reduced by 10% if grade 2 toxicity was noted, and by 20% if grade 3 or higher toxicity occurred. Treatment was delayed by one week if the neutrophil count was<3000/mm³ or platelet count <100,000/mm³ the day treatment was due. The treatment protocol was scheduled to continue until 8 weeks beyond complete remission, or until cancer progression, or until unacceptable toxicity was noted.

Physical exam, medical history, and CBC were obtained weekly. Monthly evaluation included CBC, serum biochemical profile, urinalysis, urinary tract ultrasound, and mapping of the bladder masses by ultrasound. Urinary tumors were measured by a single ultrasound operator, and estimated tumor volume was recorded. Prior to treatment and at 8-week intervals, complete tumor staging (thoracic radiography, full abdominal ultrasonography) was performed, and tumor stage determined by the World Health Organization criteria for canine urinary bladder tumors. Tumor response was defined as: complete remission (no cancer detected), partial remission (PR, ≧50% decrease in tumor volume and no new tumor lesions), stable disease (SD, <50% change in tumor volume and no new tumor lesions), and progressive disease (PD, ≧50% increase in tumor volume or the development of new tumor lesions).

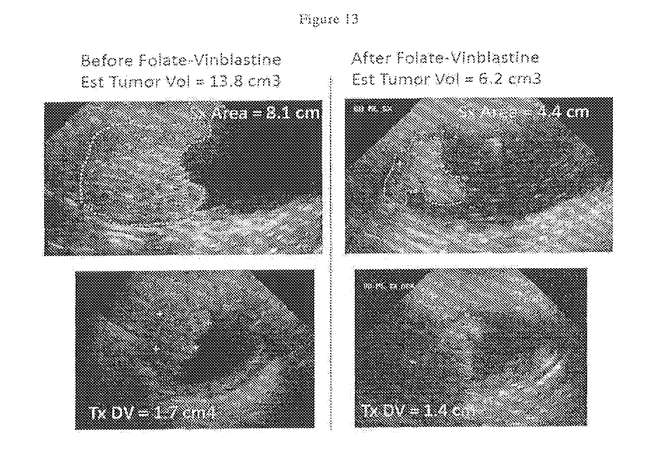

Partial remission (PR, ≧50% decrease in tumor volume and no new tumor lesions) was observed in 5 dogs (50% or cases). Stable disease (SD, <50% change in tumor volume and no new tumor lesions) was observed in 5 dogs (50% of cases). FIG. 13 shows bladder mapping with ultrasonography before (left panels) and after (right panels) EC0905 treatment.

What is claimed is:

1. A method of treating a urinary bladder cancer, comprising:
administering to a patient having urinary bladder cancer a therapeutically effective amount of a compound of the formula 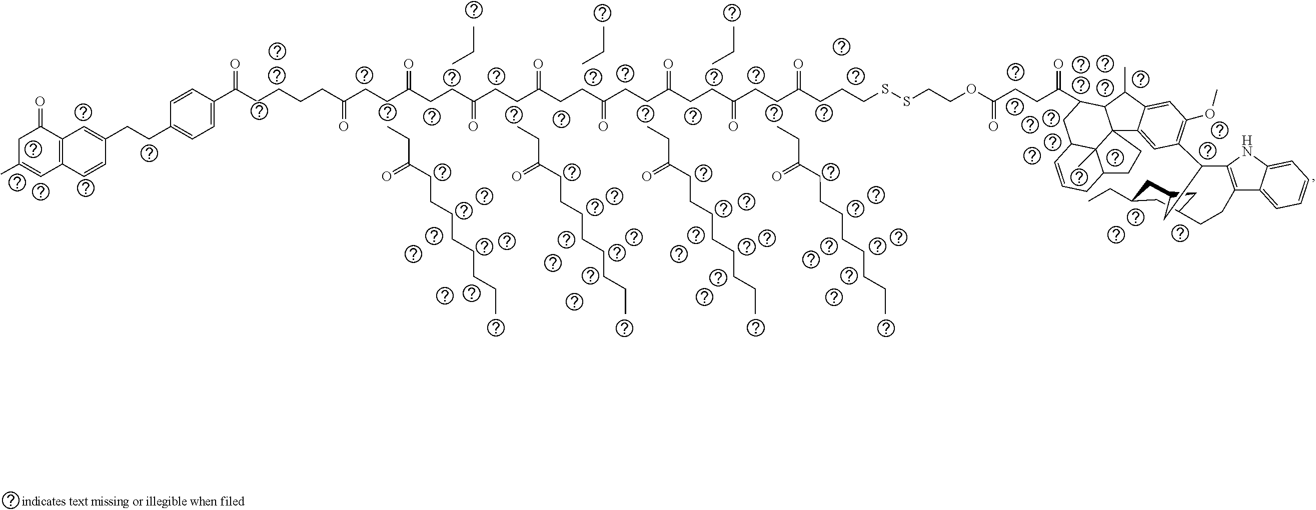
or a pharmaceutically acceptable salt thereof.

2. The method of claim 1, wherein the compound is in a composition and the composition comprises a pharmaceutically acceptable carrier.

3. The method of claim 2, wherein the composition is an inhalation dosage form, an oral dosage form, or a parenteral dosage form.

4. The method of claim 3, wherein the composition is a parenteral dosage form.

5. The method of claim 4, wherein the parenteral dosage form is an intradermal dosage form, a subcutaneous dosage form, an intramuscular dosage form, an intraperitoneal dosage form, an intravenous dosage form, or an intrathecal dosage form.

6. The method of claim 2, wherein the pharmaceutically acceptable carrier comprises a liquid.

7. The method of claim 6, wherein the liquid is saline, glucose, an alcohol, a glycol, an ester, an amide, or a combination thereof.

8. The method of claim 2, wherein the composition is in the form of a solid.

9. The method of claim 2, wherein the purity of the compound is at least 90% based on weight percent.

10. The method of claim 2, wherein the purity of the compound is at least 95% based on weight percent.

11. The method of claim 2, wherein the purity of the compound is at least 98% based on weight percent.

12. The method of claim 2, wherein the purity of the compound is at least 99% based on weight percent.

13. The method of claim 1, wherein the urinary bladder cancer is invasive transitional cell carcinoma (InvTCC).

14. The method of claim 1, wherein the pharmaceutically acceptable salt is a sodium salt.

15. The method of claim 2, wherein the composition is administered in a single dose.

16. The method of claim 2, wherein the composition is administered in multiple doses.

17. The method of claim 16, wherein the multiple doses are administered daily.

18. The method of claim 16, wherein the multiple doses are administered in a staggered regimen one to five days per week.

19. The method of claim 16, wherein the composition is administered in about 2 to about 50 doses.

20. The method of claim 16, wherein the composition is administered at 12 to 72 hour intervals.

21. The method of claim 16, wherein the composition is administered at 48 to 72 hour intervals.

22. The method of claim 1, wherein the therapeutically effective amount is about 1 ng/kg to about 1 mg/kg.

23. The method of claim 1, wherein the therapeutically effective amount is about 1 μg/kg to about 500 μg/kg.

24. The method of claim 1, wherein the therapeutically effective amount is about 1 μg/kg to about 100 μg/kg.

25. An immunohistochemical method for detecting folate receptors in urinary bladder cancer cells, comprising:
contacting the urinary bladder cancer cells with an antibody having binding specificity for a folate receptor; and
detecting folate receptor expression on the urinary bladder cancer cells,
wherein the urinary bladder cancer cells are invasive transitional cell carcinoma cells (InvTCC).

26. The immunohistochemical method of claim 25, wherein the antibody is a polyclonal antibody.

27. The immunohistochemical method of claim 25, wherein the antibody is a monoclonal antibody.

28. The immunohistochemical method of claim 26, wherein the antibody has binding specificity for the folate receptor-α.

29. The immunohistochemical method of claim 27, wherein the antibody has binding specificity for the folate receptor-α.
